(19) United States
(12) Patent Application Publication
(10) Pub. No.: US 2013/0071902 A1
(43) Pub. Date: Mar. 21, 2013

(54) METHOD AND SYSTEM FOR COLLECTING WATER FROM AN AQUATIC CELL

(71) Applicant: Tony A. Hagen, Sioux Falls, SD (US)

(72) Inventor: Tony A. Hagen, Sioux Falls, SD (US)

(73) Assignee: AQUATECH BIOENERGY LLC, Sioux Falls, SD (US)

(21) Appl. No.: 13/236,797

(22) Filed: Sep. 20, 2011

(51) Int. Cl. C12P 7/06; C12N 5/04; C12M 3/00

(52) U.S. Cl. 435/161; 435/289.1; 435/410; 435/292.1

(57) ABSTRACT

Methods and systems for collecting, purifying, and/or extracting ethanol produced during anaerobic metabolism by aquatic plants is provided. The system includes a cell containing water and an aquatic plant, an ethanol extraction assembly in fluid communication with the cell for removing ethanol from the water. Ethanol is released by the aquatic plant by initiating an anaerobic process in the plant such as by regulating the photosynthesis inducing light that reaches the aquatic plant.

### FIELD OF THE DISCLOSURE

The disclosure relates to cells for growing aquatic plants and methods and systems for collecting water from such the cells. Various aquatic plant byproducts, including ethanol may be removed from the collected water.

### BACKGROUND OF THE DISCLOSURE

Current ethanol production processes rely primarily on the direct conversion of biomass sources into ethanol. In grain based ethanol production, for example, a grain such as corn is mechanically, thermally and/or chemically processed, and a fraction extracted from the processed grain is placed in fermentation tanks containing microbes. The fermented extract is then distilled.

Drawbacks to conventional ethanol production include high raw material (i.e., grain) consumption, by-product production and consumption of water and energy. Accordingly, alternatives to convention ethanol product have been sought.

### SUMMARY OF THE DISCLOSURE

One embodiment is a cell including water, a substrate, at least one aquatic plant, a water inlet and a water outlet. At least one of the water inlet and the water outlet is positioned in the cell at or below the depth of the substrate and the other of the water inlet and the water outlet positioned in the cell at or above the depth of the substrate. Water that is removed from the cell through the outlet flows or is drawn through the substrate prior to being removed.

Another embodiment is a cell including water having an upper strata at a first water temperature and a lower strata at a second temperature, wherein the second temperature is lower than the first temperature. The cell further includes at least one aquatic plant, a water inlet positioned at the depth of the lower strata, and a substrate disposed at or below the lower temperature strata. The substrate may include an upper soil layer and a lower layer including at least one particulate material.

A further embodiment is a method of collecting water from an aquatic cell including water, a particulate substrate, and at least one aquatic plant having at least a root portion disposed in the substrate. Water is drawn through the substrate to a water outlet positioned at or below the substrate, which is connected to a water collection assembly.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawing wherein:

FIG. 1 is a schematic view of a system for growing aquatic plants according to an embodiment of the disclosure.

FIG. 2 is a cross-sectional view of a substrate for growing aquatic plants in an embodiment of the disclosure.

FIG. 3 is a cross-sectional view of a substrate for growing aquatic plants in an embodiment of the disclosure.

FIG. 4 a schematic view of a system for growing aquatic plants according to an embodiment of the disclosure.

FIG. 5 is a schematic view of a system for isolating ethanol from aquatic plants according to an embodiment of the disclosure.

FIG. 6 illustrates a method of obtaining ethanol produced by aquatic plants according to an embodiment of the disclosure.

### DETAILED DESCRIPTION

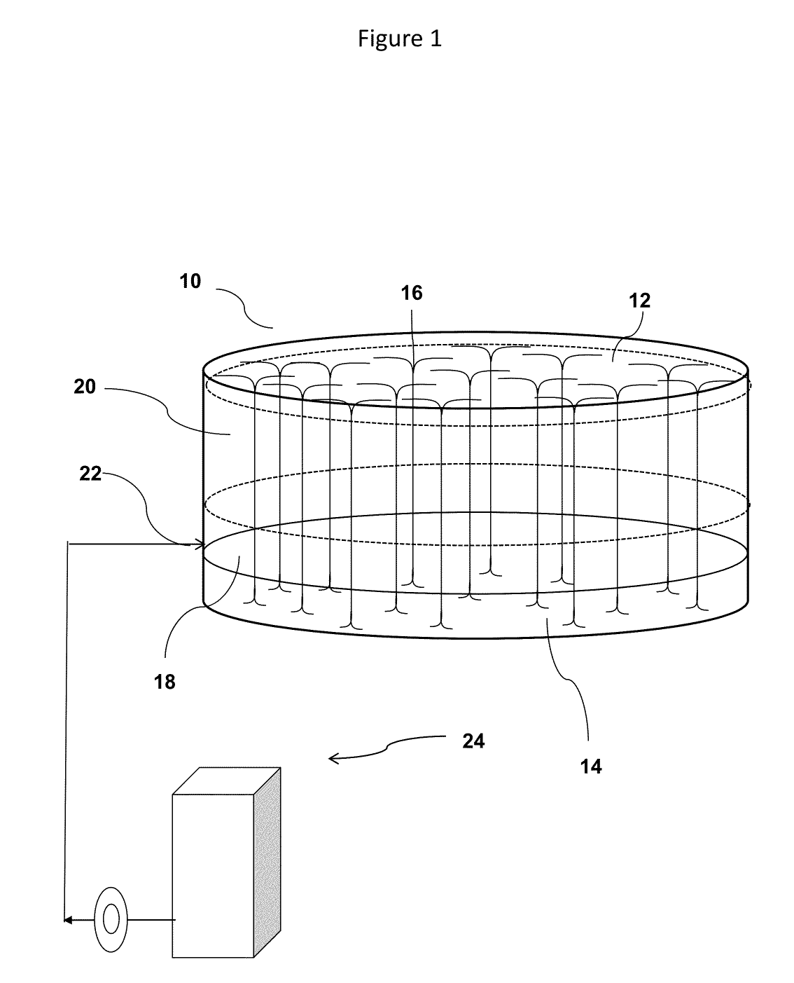

FIG. 1 is a schematic illustration of an aquatic cell 10 containing water 12, a substrate 14 disposed generally below the water near the bottom of the cell 10 and one or more aquatic plants 16.

The dimensions of cell 10 may depend on the size and type and number of aquatic plants introduced to the cell, the water depth and the substrate height. The depth of each cell can range from about 10 cm to about 20 m (e.g., 10 cm to 100 cm, 50 cm to 1 m, 100 cm to 1 m, 500 cm to 3 m, 1 m to 5 m, 4 m to 10 m, 5 m to 7 m, 5 m to 10 m, or 10 m to 20 m).

A wide range of water depths may be utilized. It has been found that some plants may grow in dramatically deeper depths providing other environmental factors, such as atypically high water temperatures at depth, are present. For instance, Stuckenia pectinata has been shown to grow in depths of greater than 20 m of water where thermal vents provide at least warmer water than would be typically found in a North American lake at such depths. In other embodiments, significantly smaller water depths may be employed.

The width and length of a cell is not crucial to the system. It is to be understood that the cell width and length need not be equal, and can be adjusted to accommodate the number and type of plant to be used in the system, and can further depend on the cell shape, available land area, access to raw materials, and cost controls. When a cell is dimensioned to hold a single plant, it may be advantageous to include more than one cell in the system.

The water 12 contained in cell 10 may be temperature controlled using, for example, heat exchangers or in-cell heaters. Heat for cells may also be sequestered from waste heat emitted by adjacent ethanol processing plants or any other convenient source of waste heat. Additional heat sources, such as geothermic and solar, may also be utilized where convenient. In one embodiment, water exiting a waste water treatment plant or electricity facility may be utilized both to regulate temperature and to provide additional nutrients to the aquatic plants. Additionally, in particularly hot climates, the cells may require cooling to prevent temperatures that would otherwise harm the plants. Depending on the variety of aquatic plant being utilized, a temperature range may be selected which optimizes plant growth and ethanol production. For example, some selected plants such as Stuckenia pectinata may be maintained between about 32° C. and 23° C. for optimal growth, though it should be understood that the overall temperature range for growth and production of ethanol falls into a much wider range. One manner of controlling temperature is to sink the cell into the ground where the soil around the cell will moderate the temperature of the cell.

In some embodiments, the temperature of the water in the cell is controlled so that regions of different water temperatures exist at different water depths. In particular, the temperature of the water can be stratified into two or more (e.g., 3, 4, or 5) generally horizontal temperature strata, such as temperature strata 18 and 20 shown in FIG. 1. The temperature in each strata can be generally uniform or may reflect a gradient.

The boundaries between strata can be maintained with or without a physical barrier. In one embodiment a temperature difference between strata 18, 20 of at least about 1° C. creates a non-barrier boundary. Minimizing water movement and temperature fluctuations in each temperature region may provide an advantage for maintaining a temperature difference between the strata without the use of a physical barrier. However, some water flow or circulation can be used to stabilize the boundaries between temperature regions. Alternatively or additionally, the stratification can be stabilized using a physical barrier, such as a cloth, mesh, or other material through which plants can grow. In some embodiments, a physical barrier is used when a temperature difference between regions is more than about 1° C. In addition, a physical barrier may reduce the amount of radiant energy (e.g., from a light source) transferred to water in a temperature region. In some embodiments, the physical barrier is put in place prior to establishment of the plant to allow the plant to grow through the barrier.

Where the water temperature is stratified, the temperature regions can be adjusted in size, location, and temperature to suit the aquatic plant being used. For example, as shown in FIG. 1, strata 20 extends from the surface of the water 12 where a leafy portion of plants 16 reside to a depth where a stem portion of the plants 16 reside. Strata 18 extends from the strata boundary at least to the substrate surface and possibly into the substrate 14 where the lower stem and/or roots of plants 16 reside.

The upper strata 20 may have a temperature of up to about 37° C. more particularly up to about 32° C., and even more particularly up to about 31° C. (including from about 2° C. to about 21° C., from about 2° C. to about 16° C., from about 4° C. to about 18° C., from about 4° C. to about 10° C., from about 10° C. to about 13° C., from about 16° C. to about 20° C., from about 17° C. to about 19° C., about 17° C., about 18° C., about 18° C., about 19° C., or the like). The temperature of the water in lower strata 18 may be from about 1-14° C., lower than the temperature of the upper strata 20, more particularly about 2-6° C. lower than the temperature of the upper strata 20. Each strata can be generally uniform in temperature or it can gradually decrease in temperature with greater depth.

Temperature in each strata may be controlled using any appropriate means. For example, in some embodiments, the water near the surface of the water is heated by exposure to radiant heat from a light source, such as the sun or an artificial light source. Alternatively, the water near the surface of the water is heated using a heat source such as a heating element or water can be heated outside of the cell and introduced to the cell near the surface. The temperature in cooler temperature regions can be controlled by, for example restricting exposure to radiant energy sources (e.g., light) through the use of physical barriers or other means, such as dye (e.g., blue dye), introducing water at a controlled temperature, or the like.

In one embodiment, the cell 10 includes at least one water inlet each disposed at a depth of one of the strata. The water inlet is attached to a water source that introduces temperature controlled water to the strata. In cell 10 illustrated in FIG. 1, a water inlet 22 is disposed within the depth of the lower strata 18. The water inlet 22 is connected to a water source 24 and is configured to deliver water to the strata 18 at a temperature that is lower than the temperature of upper strata 20, and more particularly, at a temperature at or near the desired temperature for the lower strata 18. In this manner the cooler temperature of water strata 18 is maintained and water strata 18 and 20 exist without the use of a barrier. In another embodiment, water inlets corresponding to each strata are use to independently control water temperature.

In addition to the temperature strata, other water conditions, (e.g., dissolved gas levels, nutrient levels, water flow, and the like) can be varied in the strata 18, 20 or other regions to benefit plant growth, carbohydrate production, and/or ethanol production. For example, water conditions at or near a leafy area of plants 16 can include the addition of dissolved CO₂ to promote carbohydrate production. CO₂ concentrations can be less in water regions corresponding to a stem or root region of plants 16. In another example, the flow rate of the water at or near the leafy area of an aquatic plant can be adjusted to prevent algae deposition on the leaves.

In another example, the nutrient content may differ within strata or other regions of the cell 10. Nutrient content of the water in a region corresponding to a stem area of may contain higher concentrations of macronutrients and/or micronutrients than water in a region corresponding to the leaves. In some embodiments, water near the surface of the substrate can have a higher concentration of nitrogen than water in other regions. Nutrient concentrations can be adjusted to provide nutrients to the aquatic plants as well as keep algae growth low.

The substrate 14 anchors the root system of the plants 12, and as further discussed below, may comprise a region into which plant by-products such as ethanol are released. In one embodiment, the substrate includes a particulate material that serves as the primary anchoring mechanism. However, mechanical anchoring devices such as grids or screens, to which the roots may engage and couple themselves may be optionally used as well. The ratio of water depth to substrate thickness may range from about 2:1 to about 1:2. In one embodiment, the water depth may be less than or equal to the substrate thickness, for example in a water depth/substrate thickness ratio of about 1:1 or less, more particularly from about 1:1 to about 1:2. In a further embodiment, the water depth is less than the substrate thickness, for example in a water depth/substrate thickness ratio of less than 1:1.

In one embodiment, the substrate 14 may use a coarse particulate formed from a porous mineral material. In other embodiments, the substrate 14 may include two or more materials that may be formed as layers. The characteristics of each substrate layer can be configured as appropriate for the plant being used, including variations in chemical composition (e.g., nutrient content or pH), physical composition (e.g., coarseness or density), biological composition (e.g., bacteria), and the like.

In some embodiments, one layer of the substrate 14 includes a soil composition, which may include humus, that has the ability to store and release nitrates into the water. another layer of the substrate 14 includes a porous material suitable for the colonization of bacteria, such as nitrogen-fixing bacteria. Other characteristics may also be considered, such as the ability of a substrate material to allow water, heat, gases, and/or nutrients to permeate. Additionally or alternatively, the substrate may include a layer of larger particulate material such as pea gravel, which may allow for the flow of water through the substrate.

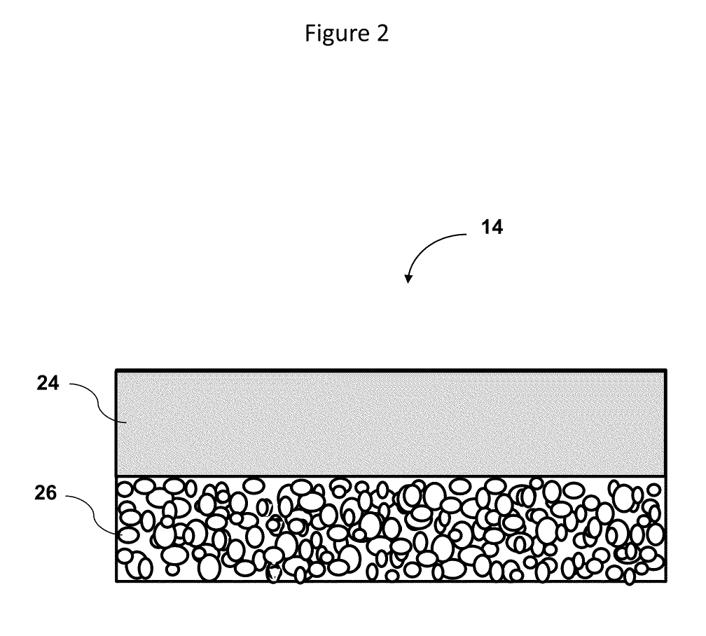

FIG. 2 is a cross-sectional view of substrate 14 according to one embodiment. Substrate 14 includes an upper layer 24 of soil or humus that extends from the surface of substrate 14 to depth of about 15 cm to about 30 cm (e.g., from about 15 to about 25 cm, from about 20 cm to about 30 cm, from about 25 cm to about 30 cm, about 15 cm, about 18 cm, about 20 cm, about 25 cm, and the like). Upper layer 24 may provide nutrients to plant roots and/or may leach nutrients such as nitrate into the water above substrate 14. Lower layer 26 is a coarse particulate material such as pea gravel suitable for allowing water flow through the substrate for reasons discussed further herein. The lower layer 26 may also encourage heat conduction if a heating element is disposed below the substrate. Lower layer 26 may have a depth of from about 15 cm to about 30 cm (e.g., from about 15 to about 25 cm, from about 20 cm to about 30 cm, from about 25 cm to about 30 cm, about 15 cm, about 18 cm, about 20 cm, about 25 cm, and the like).

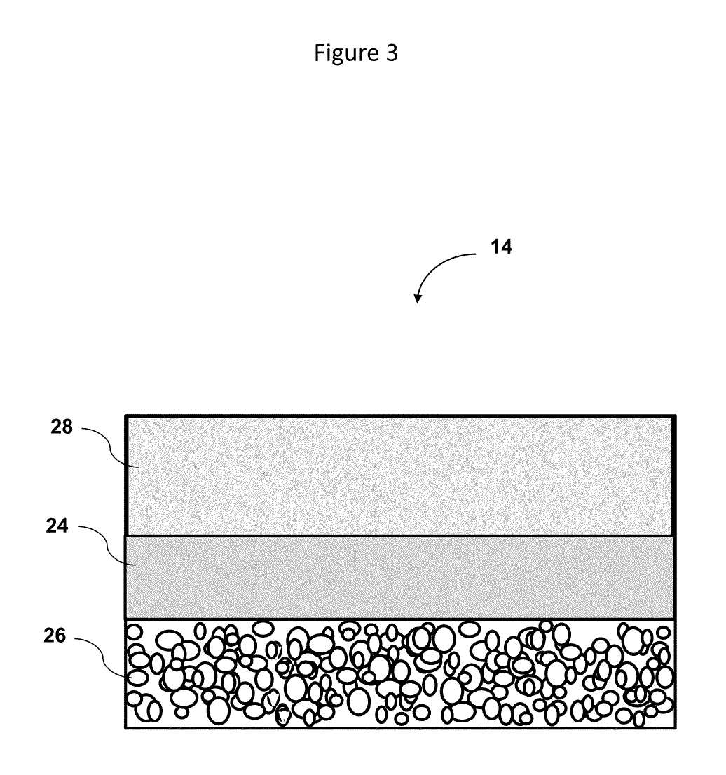

FIG. 3 is a cross-sectional view of substrate 14 according to another embodiment, which includes an additional layer 28 including a porous mineral material (e.g., Montmorillonite, calcined hematite). The additional layer 28 may provide attachment sites for nitrogen-fixing aerobic and anaerobic bacteria. The additional layer 28 is illustrated as an upper most layer of the substrate 14 and may have a height of about 15 cm to about 30 cm (e.g., from about 15 to about 25 cm, from about 20 cm to about 30 cm, from about 25 cm to about 30 cm, about 15 cm, about 18 cm, about 20 cm, about 25 cm, and the like). In alternate embodiments, layer 28 may be provided as an intermediate layer between layers 24 and 26.

In some embodiments, nutrients are added to the substrate to provide macronutrients and/or micronutrients to the plants and/or nitrogen-fixing bacteria. Nutrients can be added to the substrate using any appropriate method, such as the addition of pelletized fertilizer or nutrient-rich water through, for example, water inlet 20.

The aquatic plant 12 may be selected from any number of aquatic plants which readily live in or on an aquatic environment such as directly in water or in permanently saturated soil. More generally, the term “aquatic plant” may include any algae, aquatic plant or semi-aquatic plant which has a high tolerance for either being constantly submerged in water or intermittently submerged during periods of flooding. Further, more than one type of aquatic plant may be used within a single cell.

More, particularly, suitable plants include those that excrete ethanol under the conditions described herein. In some embodiments, the aquatic plants 16 are non-genetically modified plants. In other embodiments, the aquatic plants 16 are genetically modified plants. Genetic modifications can include, without limitation, the inclusion of a transgene that confers resistance to a pest, resistance to a pesticide or herbicide, tolerance to heat, tolerance to cold, and/or tolerance to high concentrations of plant byproducts (e.g., ethanol).

Suitable aquatic plants may include, for example, algae, submersed aquatic herbs such as, but not limited to, Stuckenia pectinata (formerly known as Potamogeton pectinatus and commonly called Sago Pondweed), Stuckenia vaginata, Stuckenia filiformis, Potamogeton crispus, Potamogeton distintcus, Potamogeton nodosus, Ruppia maitima, Myriophyllum spicatum, Hydrilla verticillata, Elodea densa, Hippuris vulgaris, Aponogeton boivinianus, Aponogeton rigidifolius, Aponogeton longiplumulosus, Didiplis diandra, Vesicularia dubyana, Hygrophilia augustifolia, Micranthemum umbrosum, Eichhornia azurea, Saururus cernuus, Cryptocoryne lingua, Hydrotriche hottoniiflora, Eustralis stellata, Vallisneria rubra, Hygrophila salicifolia, Cyperus helferi, Cryptocoryne petchii, Vallisneria americana, Vallisneria torta, Hydrotriche hottoniiflora, Crassula helmsii, Limnophila sessiliflora, Potamogeton perfoliatus, Rotala wallichii, Cryptocoryne becketii, Blyxa aubertii and Hygrophila difformmis, duckweeds such as, but not limited to, Spirodela polyrrhiza, Wolffia globosa, Lemna trisulca, Lemna gibba, Lemna minor, and Landoltia punctata, water cabbage, such as but not limited to Pistia stratiotes, buttercups such as but not limited to Ranunculus, water caltrop such as but not limited to Trapa natans and Trapa bicornis, water lily such as Nymphaea lotus, Nymphaeaceae and Nelumbonaceae, water hyacinth such as but not limited to Eichhornia crassipes, Bolbitis heudelotii, and Cabomba, and seagrasses such as but not limited to Heteranthera zosterifolia, Posidoniaceae, Zosteraceae, Hydrocharitaceae, Cymodoceaceae, and hybrids of such plants. Moreover, in one of the various embodiments, a host algae may be selected from the group consisting of green algae, red algae, brown algae, diatoms, marine algae, freshwater algae, unicellular algae, multicellular algae, seaweeds, cold-tolerant algal strains, heat-tolerant algal strains, ethanol-tolerant algal strains, and combinations thereof.

More particularly, the aquatic plants 16 are species (e.g., naturally occurring species), cross-breeds or hybrids from a family selected from one of Potamogetonaceae, Ceratophyllaceae, Haloragaceae, and Ruppiaceae. The Stuckenia pectinata species and cross-breeds and hybrids thereof (e.g., Stuckenia pectinata×Stuckenia vaginata, and Stuckenia filiformis×Stuckenia pectinata) are particularly suitable. Such aquatic plants may have a large Pasteur effect which increases the ratio of anaerobic CO₂ production to the aerobic CO₂ production. Typically this ratio is on the order of 1:3, but aquatic plants such as Stuckenia pectinata, may increase this ratio to 2:1.

The aquatic plants may be obtained and placed in the cell in any conventional manner such as gathering the plants from lakes or ponds, growing them in tanks or growing them directly in the cell 10. The type of water used in the cell will vary based on the plant type, but fresh water, salt water and brackish water are all suitable for various embodiments.

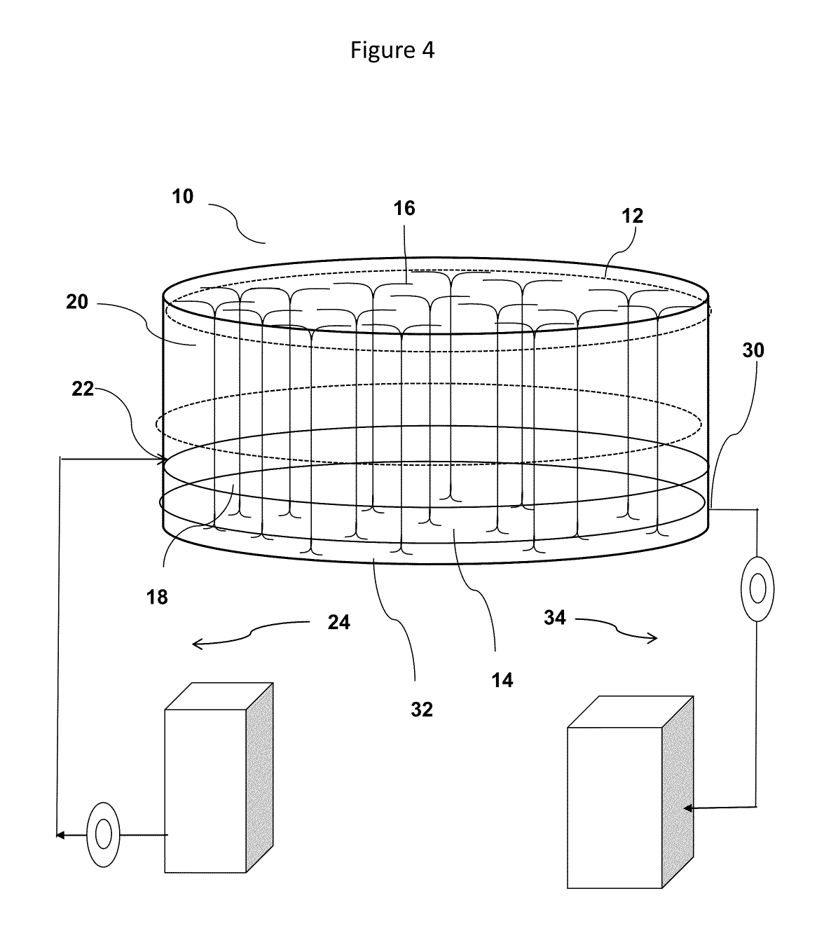

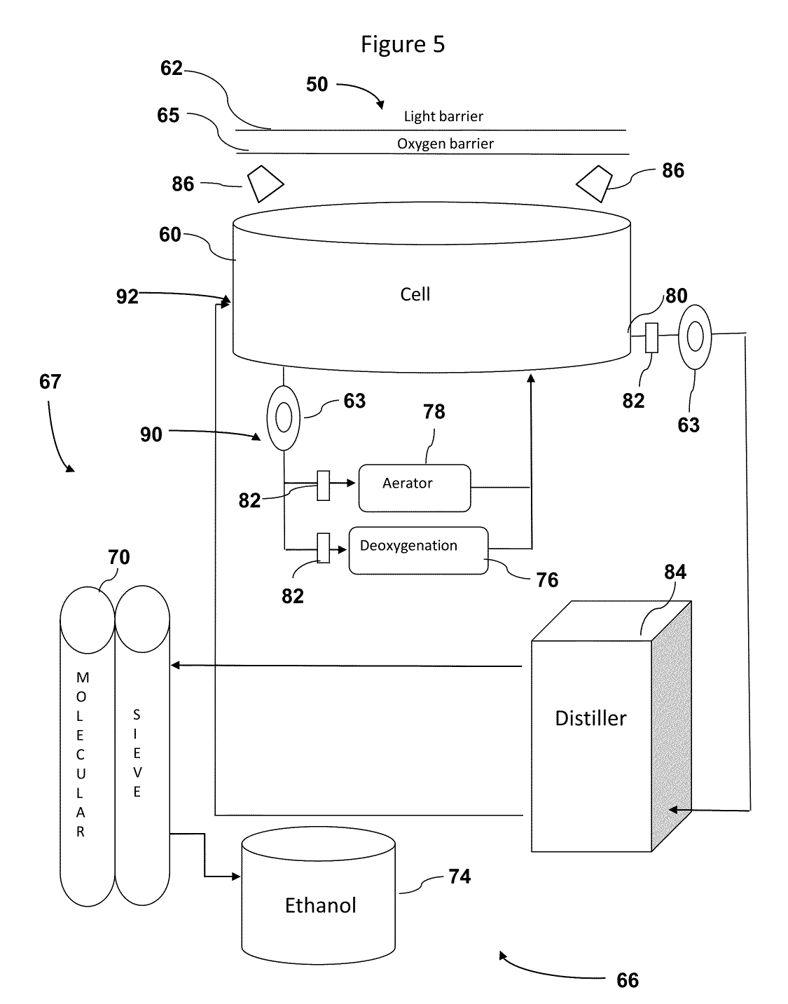

FIG. 4 illustrates another embodiment of cell 12, which further includes a water outlet 30 fluidly connected to the cell 10 at the depth of the substrate 14 or below the substrate such as in space 34. The water outlet 30 is fluidly connected to a water collection assembly 34 further described in FIG. 5. As water is drawn through the outlet 30, using a pump for example, additional water can be introduced (e.g., pumped) though a water inlet such as an opening above the water line of the cell 10 or pumped through the water inlet 22 connected to water source 24.

The water collection assembly 34 can be used to extract a variety of different components or byproducts from the water. In one embodiment, ethanol is collected from the water with an ethanol extraction assembly, which is shown and discussed in further detail with respect to FIG. 5. The experimental results set forth herein indicate that ethanol may be released by the plant into the lower strata 18 and/or substrate 14. As such, placement of the water outlet 30 at or below the substrate with the water source positioned above the substrate allows water to be drawn through the substrate region in which the ethanol (or other by-product) is most concentrated. A similar result could be obtained if the cell 12 is configured such that the water inlet 22 is disposed at or below the depth of the substrate 14 and the water outlet 30 is disposed at or above the depth of the substrate.

FIG. 5 depicts system 50 that includes a circulation loop 67 between cell 60 and ethanol removal assembly 66. The system further includes an optional circulation loop 90 having an aerator 78 and/or an oxygen removal apparatus 76 to treat water moved through the circulation loop 90 by pump 63. System 50 includes an optional artificial light source 86 that serves as a light regulating system 62 alone or in conjunction with a light barrier. In particular, artificial light source 86 may provide photosynthesis-inducing light during a light period and/or non-photosynthesis-inducing light during a dark period.

The ethanol removal assembly 66 may include a variety systems and system components that are capable of extracting and collecting ethanol from the water. In the illustrated embodiment, the assembly 66 includes one or more distillers 84 that function to separate ethanol from water. The distiller 84 is in fluid communication with one or more of a molecular sieve 70 for purifying the vapor or a condenser (not shown) for capturing ethanol vapor, and/or a container 74 to store the ethanol. A pervaporator (not shown) and/or a gas stripper could also be used if desired. For example, a gas stripper can be included in a system at a point where the concentration of ethanol is relatively low, while a distiller 84 can be included in a system at a point where the concentration of ethanol is higher. The assembly 66 allows the ethanol to be removed continuously without interrupting the processes being carried out in the cell. An ethanol removal assembly can be included in any point of a system and in any combination appropriate to remove ethanol from water in the system. In some embodiments, an ethanol removal assembly is included at multiple points in a system.

In a further embodiment, the ethanol removal assembly of any of the illustrated systems can use one or more ethanol absorptive collection systems alone or in combination with any of the other components disclosed herein. Generally speaking, ethanol absorptive collection systems utilize membrane or other absorption technology to separate ethanol from water and other extraneous materials. An example of such a membrane is the “Siftek” membrane manufactured by Vaperma Gas Separation Solutions.

Water may be drawn from and reintroduced into the cell by one or more pumps 63 through the closed loop system 67 to provide fluid communication between the cell 60 and the ethanol removal assembly. In such a closed loop system, the water outlet 80 may be disposed at or below the substrate and the water inlet 92 may be disposed at or above the substrate. This configuration allows for water to be drawn through the substrate where a significant concentration of ethanol may reside. The closed loop system 67 may include an access point to the water to allow all additives discussed above to be supplied to the water without over exposing the water to the atmosphere.

A photosynthetic light regulating system 62 is utilized to selectively allow/inhibit photosynthetic inducing light into the cell. A number of light regulation means are discussed with respect to the method 100, any of which may constitute all or a part of the light regulating system 62. For example, the light regulating system 62 can include a light-blocking cover or barrier over the cell 60. Alternatively or additionally, the light regulating system 62 includes a structure in which the cell 60 is housed or contained. It is to be understood that the light regulating system 62 can, but is not required to, inhibit all light from reaching a plant of the system. Rather the light regulating system 62 may only inhibit light at a wavelength or intensity that would induce photosynthesis in a plant of the system. For example, the light regulating system 62 can be a filter that allows only wavelengths that do not induce photosynthesis to pass. Examples of wavelengths that induce photosynthesis include wavelengths from about 380 nm to about 710 nm. Depending on the plant being used, the range of wavelengths that induce photosynthesis can be broader or narrower, but can be ascertained using known methods. In one embodiment, the sealing barrier 65 and the light regulating system 62 constitute a single structure that may or may not be separable.

The light regulating system 62 can be configured to selectively allow photosynthesis-inducing light at some time points, such as during aerobic metabolism or to induce aerobic metabolism, while inhibiting photosynthesis-inducing light at other time points, such as during anaerobic metabolism or to induce anaerobic metabolism. For example, the light regulating system 62 can be removable. In another example, the light regulating system 62 can be electrochromic, such that opacity or color of the apparatus can be controlled by the application of electric current. In some embodiments, the light regulating system 62 can include an artificial light source 86 to provide photosynthesis-inducing light and/or light that does not induce photosynthesis. Such an artificial light source 86 can be configured to emit light at an intensity or spectrum appropriate for the desired condition. For example, an artificial light source 86 can emit light at low intensity or having a wavelength outside of the range of photosynthesis-inducing light for a plant of the system during a period of anaerobic metabolism or to induce anaerobic metabolism. Similarly, artificial lighting can emit light at an intensity or at a wavelength for photosynthesis induction during aerobic metabolism of a plant of the system or to induce aerobic metabolism.

It will be evident that the various components of the cells and systems illustrated in the Figures and described herein can be used in various combinations to carry out the method 100. Additionally, conventional components can be included for controlling water flow, removing particulates, monitoring and/or maintaining water parameters (e.g., pH), monitoring ethanol concentration, monitoring and/or maintaining plant parameters, cutting, damaging or removing plants, and the like. For example, exemplary systems may include components such as valves 82, filters 80, light sensors and/or meters (e.g., photosynthetically active radiation sensor), pH meters, and the like.

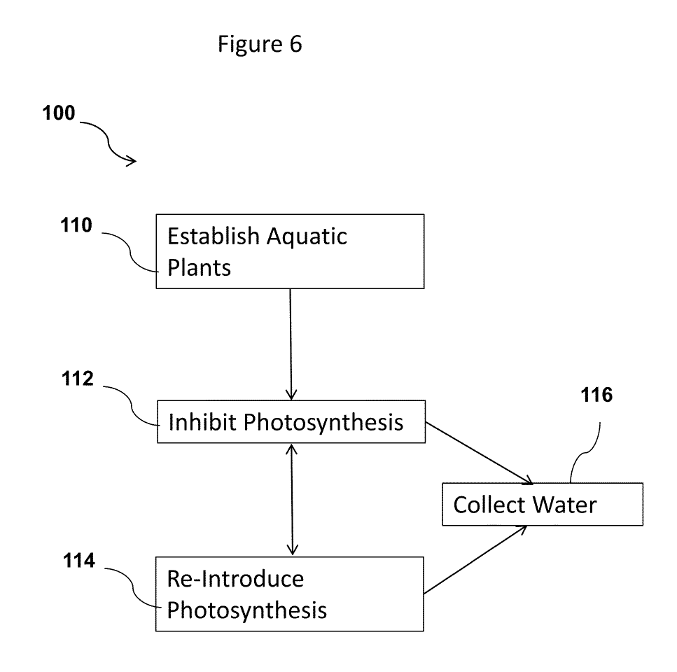

FIG. 6 illustrates a method 100 for forming and collecting ethanol according to one embodiment of the present invention, in which aquatic plants (in the form of seeds, tubers, plants, etc.) are introduced into a cell (Block 110). Once the aquatic plants are established in the cell, photosynthesis in the plants is inhibited, which results in the plants releasing ethanol into the water (Block 112). Optionally, the process may be repeated by encouraging photosynthesis in the plants through the re-introduction of photosynthesis inducing light and/or oxygen in the cell (Block 114) followed by inhibiting photosynthesis once again. Water (or byproducts contained in the water) is removed from the cell (Block 116) as desired.

If planted as seeds or tubers, it may take anywhere from 14 days to 12 months under suitable growth conditions for the aquatic plants to be sufficiently mature and viable to withstand the processing steps illustrated in FIG. 6. For Stuckenia pectinata, it may take from 5 months to 8 months for the plants to be viable.

Photosynthesis may be inhibited by shielding the plants from light sources which encourage photosynthesis. As further demonstrated in the examples below, this dark phase encourages the release of ethanol and prevents the formation of oxygen through photosynthesis. The light may be regulated by any conventional method to create dark conditions within the cell. It should be understood that the term “light” which should be blocked only applies to those forms of radiation, or wavelengths of light, which act as a photosynthesis catalyst and is dependent upon the type of chemical receptors used by each plant. Therefore, the term “dark” as used herein is meant to denote the substantial absence of the frequencies of light which promote photosynthesis.

Various means for regulating (e.g., selectively blocking/allowing) photosynthesis inducing light to reach the aquatic plant may be utilized. Such means include, for instance, barriers, covers, domes or other enclosure structure, which serve as a light barrier at least during the anaerobic process. These aforementioned barriers, covers, etc., may be removable when it is no longer desired to maintain the aquatic plant in an anaerobic condition. In one embodiment, the cells are illuminated by light visible to humans but which facilitates the “dark” condition for the plant. Other suitable regulation means include light filters that diffuse photosynthesis inducing light. Artificial lights sources may be used to preserve the dark condition and/or to selectively allow photosynthesis when the anaerobic condition is not desired. In some embodiments, a gradual transition from “light” conditions to “dark” conditions and/or vice-versa is desirable to reduce the risk of shocking the plant.

Optionally, in conjunction with the dark phase, the oxygen content of the cell can be reduced by introducing water into the cell that is severely depleted (i.e. rendered anoxic) of oxygen through the use of organic, chemical, or mechanical means. This may also be accomplished by removing oxygen from water contained in the cell. It should be understood that the term “anoxic” does not necessarily indicate a complete absence of oxygen in the water, as a very small quantity of oxygen will likely be dissolved in the water.

Alternatively or additionally, oxygen reducing additives such as corn, yeast, bacteria (e.g., genetically altered bacteria and/or bacteria capable of fermentation), or enzymes, which consume oxygen and sugars while producing carbon dioxide, may be added to the cell to deplete the oxygen levels. In order to promote the depletion of oxygen levels, a secondary carbohydrate source, for instance corn, molasses, wheat or other sources of sugar, may be added to the water for use by the oxygen reducing additives. The secondary carbohydrate source may be added along with yeast to cause a strong enough reaction to remove a significant amount of oxygen from the system. One benefit of the reduction of oxygen may be additional production of ethanol by the oxygen reducing additives.

The foregoing process causes the aquatic plants to metabolize carbohydrates and to produce ethanol. The production of ethanol may be further encouraged by the introduction of chemical catalysts and CO₂. Suitable chemical catalysts include acetic acid and 2,4-dichlorophenoxyacetic acid (known generically as 2,4d). CO₂ may be obtained from waste sources such as electricity facilities and petroleum refineries. Additional nutrients and salts such as salts of potassium, nitrogen and phosphorus may further be added to promote growth of the aquatic plants. Further, depending upon the species of aquatic plant being utilized, organic substrates, including but not limited to those such as sucrose, glucose and acetate, may also be added to the cell.

Photosynthesis may be inhibited in the plants from one to several days. In the case of Stuckenia pectinata photosynthesis may be inhibited from 1 to 14 days, more particularly, from 2 to 10 days, and even more particularly, from 3 to 7 days. The time required will depend on many factors such as light diffusion, availability of nutrients, size of the cell, size of the plant, plant variety and carbon content of the plant. The determination of length of time is primarily dependent upon maximizing output of ethanol while still allowing for plant recovery by reintroducing photosynthesis. When the plant decreases its ethanol production beyond useful parameters, there may be no need to retain it in the anoxic conditions. Further, the pH of the cell must be monitored to prevent the water from becoming too acidic or basic. This may be counteracted with calcium buffering compounds such as calcium carbonate and calcium chlorate or by introducing CO₂ (to basic water) or depleting CO₂ to raise pH (e.g., by stripping or photosynthesis-driven depletion), but will ultimately be dependent upon the tolerances of the particular aquatic plant species in the cell. In some embodiments, a drop in intracellular pH (e.g., a drop of about 0.2 pH units) may trigger ethanol formation. pH can be raised just prior to ethanol formation induction to prevent the pH drop from exceeding plant tolerance and/or intracellular acidosis.

In further embodiments, the anaerobic process may be facilitated by covering the cells with one or more sealing barriers to regulate the movement of gasses (e.g., air, oxygen, CO₂, nitrogen, etc.) into and out of the cell. For example, a sealing barrier may prevent the unwanted introduction of oxygen into the cell. The sealing barrier (or an additional sealing barrier) may also be used to retain CO₂ within the cell, particularly if CO₂ is being added to the cell. Additionally, high N₂ levels may be maintained as well to further dilute any O₂ within the water or trapped between the seal and the cell. The sealing barrier would seal the cell to prevent fluid communication between the cell and the adjacent atmosphere. This will inhibit oxygen from entering the cell and will encourage the anaerobic process. In some embodiments, the sealing barrier may also facilitate the maintenance of humidity levels above the surface of the water to prevent drying out of immersed leaves. In addition, leaves may be sprayed or misted with water to prevent drying. The sealing barrier may be a translucent barrier to encourage the capturing of radiant heat from a light source which is either naturally and/or artificially used to provide light to the aquatic plants. The sealing barrier may or may not also constitute a light blocking barrier which, as discussed above, is positioned on the cell to prevent light from entering the cell during the anaerobic process. The sealing and light blocking barriers may be made of conventional materials. However, it should be understood that a dwelling, tank, dome or other structure constructed around the cell may also define sealing and light block barriers should they be used in such a capacity.

In one embodiment, the process described above is preceded by, followed by or alternated by re-initiating photosynthesis in the plant. The aquatic plant is exposed to light to induce photosynthesis and to stop the anaerobic process by allowing an oxygenated condition within the cell, which initiates and/or facilitates the aerobic process. This light phase may be accomplished by manipulating the light regulating means and systems discussed herein. For example, a light barrier, cover, or filter etc., may be removed so that natural or artificial photosynthesis inducing light is allowed to reach the aquatic plant. Alternatively or additionally, a light barrier may remain in place and an artificial light source is regulated to allow photosynthesis inducing light to reach the aquatic plant.

During the light phase, an aerobic process may be further initiated by creating an oxygenated condition in the cell, which facilitates the production and storage of carbohydrates by the aquatic plant. This oxygenated condition may be created by a variety of approaches, which may be used independently or in combination. In one embodiment, oxygenated water is added to the cell or oxygen is directly introduced into water contained in the cell. In another embodiment, the gas barrier is removed to allow the oxygen concentration of the water to naturally increase. Accordingly, the oxygenated condition may be accomplished by introducing oxygenated water into the cell, by removing anoxic water and/or allowing the water to oxygenate naturally by plant releasing of oxygen and exposure to an oxygenated atmosphere.

During the aerobic process, nutrients may be added to the cell to provide nutrients to the aquatic plants. Additionally, maximum sunlight/artificial light filtration is encouraged as is temperature regulation to promote growth of the aquatic plants. The light itself may be intensified by the addition of artificial light.

Generally, the light phase is continued for between ½ day and 15 days, and more generally at least 3 to 10 days, to allow the aquatic plants to re-form carbohydrates, though this time frame may be adjusted for plant specific requirements. During this time the aquatic plants create and retain carbohydrates through metabolic processes. The duration of the aerobic process is dependent upon a number of factors but will typically end when carbohydrate production begins to slow or reaches a predetermined level. With Potamogeton pectinatus (Stuckenia pectinata) this may be between 2 days and 14 days, more particularly, 3 to 10 days depending upon environmental conditions within the cell As used herein, the term “day” means a 24 hour period.

It has been found in particular that manipulating light and dark conditions can affect the manner in which the aquatic plants produce ethanol and sugars. For instance, some aquatic plants may be subjected to light for several continuous days defining a light phase followed by restriction to light for several continuous days defining a dark phase to facilitate the, ethanol producing, process. In one embodiment, the dark phase is timed to occur simultaneously or shortly before or after the initiation of an anaerobic condition, preferably within 1 to 3 days of one another.

When the light phase ends, there may be a transition period between the oxygenated phase and the anoxic phase where the amount of oxygen is being depleted. During the transition period, it may be beneficial to add the yeast to the cell which will stimulate the reduction of the oxygen and will allow the yeast to produce the ethanol. The ethanol formed by the yeast may act as a catalyst for anaerobic activity by the plant and will offer an additional ethanol production outlet. Sugars or other carbohydrates added along with the yeast may further enhance anaerobic activity.

Generally, the ratio of dark phase to light phase will be no more than 1:2 and as small as 1:10, with a more common ratio of between 1:2 and 1:7. It should be understood that during both of the light and dark phases, CO₂ may be added to the water to encourage both the formation of sugar and ethanol. Finally, the ability to control the light and dark phases above and the ratios described herein are not applicable to all aquatic plants as certain plants may experience ethanol production after less than 4 hours of dark phase. For these types of aquatic plants, the ratio of light phase to dark phase may be greater than 2:1, though such aquatic plants may have different limitations with respect to ethanol production than experienced with plants such as Stuckenia pectinata.

Once maximum carbohydrate formation, or a predetermined level of such, is approached, a dark phase is again initiated to begin the process of carbohydrate metabolism and ethanol formation. The steps of inhibiting and re-introducing photosynthetic conditions can be repeated to continually promote ethanol production followed by carbohydrate production. In some embodiments, the “light” and “dark” periods may be timed or regulated in a pattern to simulate day and night conditions. It can be desirable to initiate ethanol production at the beginning of a “dark” period to maximize the availability of the carbohydrate stored during the previous “light” period to ethanol conversion.

This process creates a self-sustaining cycle as the plant growth replenishes both plant matter lost to plant senescence and those plants which no longer meet established tolerances of ethanol production. Additional plant growth which cannot be used for replenishing purposes or for furnishing plant material for another cell may be removed and fermented using conventional methods to also produce ethanol. Carbon dioxide released during the fermentation process may be captured and returned to the cell to promote carbohydrate production. Plant waste, both before or after the fermentation process, may further be used for replenishing nutrients to the cell as feed material and/or may be processed for biochemical industrial usage such as in ethanol and diesel biofuels, pharmaceuticals, cosmetics, colorants, paints and the like.

While the method 100 is being practiced, bacterial and algal blooms may occur which can be controlled by antibiotics, bi-sulfates, hops, algaecides, chlorination, ultraviolet light exposure and other common practices. Additionally, ethanol producing yeasts may be added to the cell for the purpose of decreasing the carbohydrate concentrations and inhibiting bacterial growth. Alternatively, or in conjunction with yeast, enzymes or bacteria may also be used to decrease carbohydrate concentrations. A potential beneficial of the addition of yeast is an increase in ethanol output. The yeast may require replacement, particularly after an anoxic condition has been established and maintained for more than about three days, though this is dependent upon the strain of yeast being used. A secondary carbohydrate source may also be added to the system to cause the yeast to react more strongly.

The foregoing process may be more broadly defined to include: 1) a recharge phase wherein the water is oxygenated and/or the plant is exposed to light so that carbohydrates are formed, 2) a transition phase wherein the cell is deprived of photosynthesis inducing light and/or yeast may be added to form ethanol and deplete oxygen, and 3) a fermentation phase wherein the plant releases ethanol. An optional fourth phase may be defined as a second transition phase wherein photosynthesis is reintroduced. The phases may each be modified as taught herein to maximize plant growth and ethanol output. In one method, the recharge phase may occur over 0.5-12 days, followed by 0.5-6 days of the transition phase, which is then followed by at least 6 days of anoxic phase which may be increased to more than 20 days depending on the type of plant being utilized. In another method, the recharge phase may occur over 3-10 days, followed by 2-6 days of the transition phase, which is then followed by at least 2 days of dark phase which may be increased to more than 20 days depending on the type of plant being utilized. It may be advantageous to lower water levels following the recharge phase and prior to the anoxic phase to reduce water volume and further concentrate ethanol during the anoxic phase.

At any time during the light or dark phases, water may be removed from the cell in order to extract by-products such as ethanol. In one embodiment, which may be carried out using the cells illustrated in FIGS. 4 and 5, water is removed by flowing the water through the substrate to a water outlet connected to an ethanol removal assembly. As further indicated in the examples, a significant amount of ethanol is contained within the substrate and/or lower strata region of the water. By drawing water through the substrate and into the water outlet, improved ethanol extraction efficiency may be achieved. In one embodiment, the water outlet is positioned at or below the substrate, and water is drawn through the substrate and into the outlet. Water may be introduced to the system through an inlet, including by adding water at the top of the cell.

As further indicated in the Examples, ethanol may convert to acetic acid under certain conditions. Accordingly, it may be beneficial to extract ethanol during the dark phase before acetic acid conversion begins to reduce the overall ethanol concentration. Additionally, or alternatively, cell conditions can be manipulated to limit the presence of acetobacters in the water.

### EXAMPLES

### Example 1

Two Stuckenia pectinata plants with tubers attached were removed from stock growth tanks and individually placed into a test tube with 35 ml of boiled distilled water. A Resazurin indicator was included in the water to show anoxic conditions. These anoxic samples were placed within foil wraps to produce dark conditions by preventing photosynthesis-inducing light from reaching the plants, which would allow the water within the plant cells to become re-oxygenated. The samples were then placed in a chamber with a positive pressure nitrogen atmosphere to prevent re-oxygenation of the extra-cellular sample water. The samples were then allowed to incubate in this chamber at about 24° C. for 3 days. On the morning of the fourth day a 2 ml sample of water was removed from each sample and analyzed by high pressure liquid chromatography (HPLC) at South Dakota State University to detect the presence of ethanol. HPLC peaks in each sample indicated that ethanol was present.

### Example 2

Stuckenia pectinata plant samples were taken from lake material gathered from South Dakota lakes and were placed in vials with boiled distilled water to provide anoxic conditions added only to cover plants. Eight samples, D5-8, D11, and D14-16 were placed in a sealed stainless steel pot within the incubator to provide dark conditions for the samples. The remaining samples, D1-4, D9-10, and D12-D13, were placed in clear plastic quart containers with airlocks. Antibiotic was added to samples D9-D16 to prevent ethanol conversion to acetic acid by bacteria. The samples were placed in an incubator at approximately 21° C. and allowed to incubate for 7 days. Water from each sample was drawn and analyzed by high pressure liquid chromatography (HPLC) at South Dakota State University to determine ethanol and acetic acid concentrations.

The four samples, D5, D6, D7, and D8, incubated without antibiotic in dark conditions contained ethanol at a concentration of 10.825 g/L, 6.817 g/L, 7.733 g/L, and 10.595 g/L, respectively. Samples D11 and D14, which were incubated in dark conditions with antibiotic had ethanol concentrations of 6.573 g/L and 4.237 g/L, respectively. In addition, sample D11 contained no acetic acid, while sample D14 contained acetic acid at a concentration of 2.192 g/L, suggesting that the amount of antibiotic in sample 14 was insufficient to prevent ethanol conversion to acetic acid by bacteria. The samples incubated in the clear containers contained no detectable ethanol, suggesting that photosynthesis interfered with ethanol production by the plant samples. The results are shown in Table 1.

TABLE 1

| | | | Acetic acid | Ethanol |
|---|---|---|---|---|
| Sample | Dark conditions | Antibiotic | (g/L) | (g/L) |
| D1 | − | − | 1.332 | 0 |
| D2 | − | − | 1.616 | 0 |
| D3 | − | − | 0.503 | 0 |
| D4 | − | − | 1.142 | 0 |
| D5 | + | − | 2.204 | 10.825 |
| D6 | + | − | 2.865 | 6.817 |
| D7 | + | − | 1.420 | 7.733 |
| D8 | + | − | 5.091 | 10.595 |
| D9 | − | + | 0 | 0 |
| D10 | − | + | 0 | 0 |
| D11 | + | + | 0 | 6.573 |
| D12 | − | + | 0.863 | 0 |
| D13 | − | + | 0.749 | 0 |
| D14 | + | + | 2.192 | 4.237 |
| D15 | + | + | 0.730 | 0 |
| D16 | + | + | 0 | 0 |

### Example 3

A cell having a length of about 184 cm, a width of about 46 cm and a depth of about 58 cm was filled with a substrate and water. The substrate was about 8 cm deep and the water was about 43 cm deep. The substrate included a lower layer of about 4 cm of black soil and an upper layer of commercially available agri-lime (Premium Infield from Prochoice One). Sample Ports were included at an upper portion of the tank, a middle portion of the tank and a lower portion at the substrate layer.

The cell was seeded with approximately 70 Stuckenia pectinata plants and allowed to grow for 2 months. The water was not circulated during this time. Once the plants were grown and established, 3 tablespoons of sugar was added from the top of the cell by mixing the sugar with a small volume of water from the cell and then adding that volume of water back into the cell without further mixing. A clear plastic cover was placed over the water surface to discourage evaporation, the cell was covered with light blocking plastic and the top of the tank was sealed. The tank was maintained in this manner for 6 consecutive days. Then, air was bubbled into the cell for three hours after which a portion of the light blocking plastic was removed in order to gradually re-introduce light conditions. Over time, the remaining light blocking plastic was removed.

Water samples were removed from the upper, middle and lower ports on a daily basis during the six day period in which photosynthesis was inhibited. The samples were tested for ethanol and acetic acid. The results are shown in Table 1 below in grams per liter.

TABLE 1

| | Upper | Middle | Lower | Upper | Middle | Lower |
|---|---|---|---|---|---|---|
| Day | Ethanol | Ethanol | Ethanol | Acetic | Acetic | Acetic |
| 0 | 0.000 | 0.000 | 0.000 | 0.000 | 0.000 | 0.000 |
| 1 | 0.005 | 0.000 | 0.000 | 0.000 | 0.000 | 0.000 |
| 2 | 0.000 | 0.000 | 0.031 | 0.000 | 0.000 | 0.093 |
| 3 | 0.000 | 0.010 | 0.032 | 0.000 | 0.000 | 0.269 |
| 4 | 0.011 | 0.010 | 0.027 | 0.055 | 0.056 | 0.265 |
| 5 (1) | 0.000 | 0.000 | 0.000 | 0.044 | 0.047 | 0.291 |
| 5 (2) | 0.000 | 0.000 | 0.000 | 0.044 | 0.044 | 0.222 |
| 6 | 0.000 | 0.000 | 0.000 | 0.050 | 0.050 | 0.206 |

The results from Table 1 indicate that the highest concentration of ethanol was observed in samples taken from the lower samples port at the level of the substrate. This suggests that ethanol is being released from the root/tuber regions of the plant during the first several days of the dark phase. Similarly, the highest concentration of acetic acid was observed primarily in samples taken from the lower port during the latter portion of the dark phase. This indicates that ethanol in the water was converted to acetic acid, possibly by acetobacters present in the substrate.

The addition of sugar to the cell prior to the dark phase did not appear to materially contribute to the measured concentration of ethanol. First, the sugar was added at the top of the tank without further mixing, while ethanol was observed primarily in samples taken from the lower port, indicating that the ethanol came from another source. Additionally, the measured ethanol/acetic acid concentrations were greater than the theoretical yield that could be obtained from the added sugar even assuming 100 percent conversion.

Seven days after reintroducing light conditions, 11 plant stems survived and exhibited new leave growth. Of the plants whose stems that did not survive, several showed new leave growth near the base of the plant.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure.

1. An aquatic cell adapted to remove plant by-products comprising:
water;
a substrate comprising a particulate material;
at least one aquatic plant having at least a root portion disposed in the substrate;
a water inlet adapted deliver water into the cell;
a water outlet adapted to remove water from the cell; and
an ethanol extraction assembly fluidly connected to the water outlet;
wherein at least one of the water inlet and the water outlet is positioned in the cell at or below the substrate and the other of the water inlet and the water outlet is positioned in the cell at or above the substrate such that water that is removed from the cell through the outlet is drawn through the substrate.

2. The aquatic cell of claim 1 wherein the water outlet is positioned at or below the substrate and the water inlet is disposed at or above the substrate.

3. The aquatic cell of claim 1 wherein the water outlet is positioned in a wall or floor of the cell below the substrate.

4. The aquatic cell of claim 1 wherein the water comprises an upper strata having a first temperature and a lower strata comprising a second temperature, wherein the second temperature is lower than the first temperature.

5. The aquatic cell of claim 4 wherein the water inlet is positioned within the lower strata and above the substrate.

6. The cell of claim 1 wherein a ratio of water depth to substrate height is between about 1:1 and 1:2.

7. The cell of claim 1 wherein the at least one aquatic plant is selected from one of the Potamogetonaceae, Ceratophyllaceae, Haloragaceae, and Ruppiaceae families.

8. The cell of claim 1 wherein the at least one aquatic plant is selected from the Potamogetonaceae family.

9. The cell of claim 1 wherein the at least one aquatic plant is the Stuckenia pectinata plant or a cross-breed or hybrid thereof.

10. An aquatic cell comprising:
water comprising an upper strata having a first water temperature and a lower strata having a second temperature, wherein the second temperature is lower than the first temperature;
a water inlet positioned within the lower strata;
a substrate disposed at or below the lower temperature strata and comprising an upper soil layer and a lower layer including at least one particulate material; and
at least one aquatic plant having at least a root portion disposed in the substrate.

11. The aquatic cell of claim 10, wherein the particulate material comprises gravel.

12. The aquatic cell of claim 10, wherein the substrate comprises a third layer comprising an mineral material.

13. The aquatic cell of claim 10 wherein the water inlet is fluidly connected to a water source having a temperature that is lower than the first temperature.

14. The aquatic cell of claim 10 further comprising a photosynthetic light regulator configured to selectively inhibit photosynthesis in the aquatic plant.

15. The aquatic cell of claim 10 wherein the photosynthetic light regulator comprises a photosynthetic light barrier.

16. The aquatic cell of claim 10 wherein the at least one aquatic plant is the Stuckenia pectinata plant or a cross-breed or hybrid thereof.

17. The aquatic cell of claim 10 further comprising a water outlet positioned in the cell at or below the substrate such that water that is removed from the cell through the outlet is drawn through the substrate.

18. A method of collecting water from an aquatic cell including water, a particulate substrate, and at least one aquatic plant having at least a root portion disposed in the substrate, the method comprising:
drawing water through the substrate into a water outlet in fluid communication with an ethanol collection assembly.

19. The method of claim 18 wherein the water outlet is positioned at or below the substrate and the drawing step comprises drawing the water downwardly from a region at or above the substrate into the water outlet.

20. The method of claim 18 further comprising the step of forming upper and lower water strata in the cell.

21. The method of claim 20 further comprising introducing water into the lower strata having a temperature that is lower than the water temperature of the upper strata.

22. The method of claim 18 further comprising the step of removing ethanol from water drawn into the ethanol collection assembly.

23. The method of claim 18 wherein water is drawn into the water outlet while photosynthesis of the cell is inhibited.
